(19) United States
(12) Patent Application Publication
(10) Pub. No.: US 2005/0096383 A1
(43) Pub. Date: May 5, 2005

(54) Method and composition for oral cavity hygiene

(76) Inventor: Ingvar Olafsson, Clearlake, CA (US)

Correspondence Address: MILORD & ASSOCIATES, P.C., 10880 WILSHIRE BOULEVARD, SUITE 2070, LOS ANGELES, CA, 90024, US

(21) Appl. No.: 10/702,212

(22) Filed: Nov. 4, 2003

(51) Int. Cl. A61K 31/355

(52) U.S. Cl. 514/458

(57) ABSTRACT

The present invention relates to a composition and method for treating individuals afflicted with periodontal disease, namely gingivitis, through the application of vitamin E to the periodontum.

### FIELD OF THE INVENTION

The present invention relates generally to a composition and method for treating disorders in the mouth, more specifically, the present invention relates to the treatment of gingivitis by the topical application of an effective amount of a formulation comprised solely of Vitamin E.

### BACKGROUND OF THE INVENTION

Gingivitis and periodontal disease are all undesirable conditions that effect many people. It is well known that these conditions are associated with an accumulation of dental plaque, which is formed by absorption and propagation of intraoral bacteria such as Streptococcus mutans and the like on the surfaces of the teeth. Therefore, it is important to remove dental plaque to prevent and treat the above-mentioned conditions. Several medical treatments have been used in an attempt to treat sensitive gums, gingivitis and related conditions of the periodontum of the oral cavity.

Periodontal disease is a chronic bacterial infection accompanied by inflammation that affects the gums and bone supporting the teeth. Gingivitis is the first form of periodontal disease and is caused by the long term effects of plaque deposits. Plaque is a mucopolysaccharide which forms naturally in the mouth, as a result of the presence of saliva and bacteria. At the outset of plaque formation, plaque exists as a web of soft, sticky gelatin, called the pellicle. The pellicle develops on the exposed portion of the teeth, and provides a haven for material such as bacteria, mucus, and food debris. Unremoved plaque mineralizes into a hard deposit called calculus (tartar) that becomes trapped at the base of the tooth. Plaque and calculus cause mechanical irritation and inflammation of the gingival. Bacteria, and the toxins produced by the bacteria, cause the gums to become infected, swollen and tender.

Symptoms of gingivitis include mouth sores, swollen gums, the gums appear bright red or red-purple, the gums appear shiny, the gums bleed easily and the gums are tender when touched, but can otherwise be painless. Untreated gingivitis can advance to periodontis. The plaque can spread and grow below the gum line, toxins produced by the bacteria in plaque irritate the gums. The toxins then stimulate a chronic inflammatory response in which the body in essence turns on itself, and the tissues and bone that support the teeth are broken down and destroyed. Gums separate from the teeth, forming pockets—spaces between the teeth and gums—that become infected. As the disease progresses, the pockets deepen and more gum tissue and bone are destroyed. Often this destructive process has very mild symptoms. Eventually, the teeth can become loose and may have to be removed.

As a result of the hypotheses as to the cause of periodontal disease, several treatments for the condition have been based thereon. The goal of the treatment is to reduce gingival inflammation. Treatment options include, teeth cleaning, meticulous brushing and flossing, as well as antibacterial mouth rinses and other aides. The usual approach towards combating the accumulation of dental plaque is by mechanical ways, such as brushing and flossing. Even the most thorough mechanical cleaning fails to eliminate interproximal dental plaque. Furthermore, in practice, the majority of people fail to regularly conduct a sufficient cleaning and, therefore, the rate of diseases such as gingivitis and periodontal disease is not reduced despite mechanical cleaning. It therefore becomes necessary to complement mechanical oral hygiene measures with chemotherapeutic agents to inhibit the development of dental plaque.

Other current therapies for the peridontum have their complications and drawbacks. Non-surgical treatment includes scaling and root cleaning, which is deep root cleaning of teeth to physically remove all tartar and plaque. The teeth are polished to remove stains and to make the teeth harder for the plaque to adhere to. This procedure is expensive, time consuming and the patient must adhere to follow up treatments as prescribed by their hygienist. Treatment of the oral cavity with antibiotics to reduce or eliminate the effects of bacteria is also known. For example, broad spectrum antibiotics such as tetracyclines and metronidazole have been used in the treatment of periodontal disease to reduce oral cavity microflora. Typically such use has been systemic, which can result in various undesirable side effects, including the threat or danger or building allergies or immunity to the antibiotic, overgrowth of opportunistic yeast and fungi and intestinal disturbances. However, because of the chronic nature of periodontal disease, the ideal treatment needs to be used long-term in a safe and convenient manner.

The group of tocopherols, commonly described as vitamin E, comprises several structurally closely related lipophilic vitamins, namely alpha-tocopherol, beta-tocopherol, gamma-tocopherol, delta-tocopherol, epsilon-tocopherol. Alpha-tocopherol (most commonly known as d-alpha-tocopherol and dl-alpha-tocopherol), being the most important in biological terms. The tocopherols can be found in many plant oils, especially rich in tocopherols are the seed oils of soybean, wheat, maize, rice, cotton, lucerne and nuts. Also fruits and vegetables, e.g. raspberries, beans, peas, fennel, pepper etc. contain tocophrols. As far as presently known, tocopherols are exclusively synthesized in plants and photosynthetically active organisms.

Due to their redox potential, tocopherols contribute to avoid oxidation of unsaturated fatty acids by air oxygen; alpha-tocopherol is the most important lipophilic antioxidant in humans. It is assumed that due to their function as antioxidative agents, the tocopherols contribute to the stabilization of biological membranes, because the fluidity of the membranes is maintained by the protection of the unsaturated fatty acids of the membrane lipids.

Vitamin E has demonstrated to be efficacious in treating periodontal disease. U.S. Pat. No. 3,992,519 to Hoffmann et al., assigned to Beecham Group Limited, ('519 patent), states that benefits have been claimed in the prophylaxis and treatment of inflammatory diseases of the periodontum from the application within the oral cavity of compositions containing vitamins and vitamin like materials. Accordingly, the '519 patent teaches a composition containing vitamin E for use in oral hygiene. Although the teachings may be an effective tool for oral hygiene, the vitamin E used in this composition is in a weak concentration of only 0.1% of the entire composition by weight.

U.S. Pat. No. 4,226,851 to Sompayrac ('851 patent) teaches a method for treatment of the oral cavity via a chemically stable oral hygienic composition of matter comprised of hydrogen peroxide and zinc chloride, the composition of matter containing water-soluble vitamin E in an effective amount as the stabilizing agent. Although the '851 patent discusses vitamin E being a component of the solution, the vitamin E is suggested to be at a lower 7000 I.U. concentration, is water soluble, and its purpose in the solution is to stabilize the mixture against oxygen release therefrom during storage.

U.S. Pat. No. 4,411,885 to Barels et al. ('885 patent) discloses the use of a substantially anhydrous composition having vitamin E, a surfactant agent and an abrasive agent suitable for removal of plaque in a capsule or tablet form to be chewed and digested. The '885 patent suggests a very low concentration of vitamin E (10 mg or the equivalent of 10 I.U. of vitamin E) in the composition.

U.S. Pat. No. 5,747,005 to Barels et al. ('005 patent) discloses an oil-based anti plaque which contains vitamin E. While the '005 patent teaches that the oil base should be within the rage of 55 weight percent to about 70 weight percent of the total composition, it fails to disclose a particular I.U. concentration of the vitamin E in order to be effective in the treatment of gingivitis and plaque reduction.

U.S. Pat. No. 6,432,388 to Alvarez Hernandez ('388 patent) discloses a whitening toothpaste which may contain vitamins, including vitamin E, which is usually used in the form of acetate salt, and is implied to have a calming and anti-inflammatory effect, protecting oral mucus against lipid peroxidation and favors the healing of injuries. The '388 patent incorporates several vitamins and mixtures thereof, in a proportion lying between 0.1% and 5% by weight of vitamin present with respect to the total weight of the formulation. While this patent may improve upon Spanish patent application no. P9401832, it fails to provide any concrete evidence that the low concentration of vitamin E in this composition may be effective in the treatment of gingivitis and periodontal disease.

Pure vitamin E at a concentration greater than 7,000 I.U. has been used as a cosmetic application as a moisturizer for dry skin areas and to minimize the appearance of wrinkles and stretch marks. Further, said concentrations generally indicate a warning of “FOR EXTERNAL USE ONLY.” Such high I.U. concentrations used orally are known to cause an oily sensation or unpleasant mouth feel. As can be seen in the aforementioned patents, previous attempts to use vitamin E have been at low concentrations and vitamin E at greater concentration levels have born specific instructions not to use the same orally.

Treatment regimens for periodontal disease typically involve removal of mechanical irritants, such as plague and calculus, and removal of as many bacteria as possible. The tissues are made as cleansable and as healthy as possible. The patient is taught to keep teeth and gums clean. Thus, a need exists for a composition and method for treating the peridontum without causing the shortcomings of the prior art. In addition, a need exists for a composition and method for treating the periodontum that is more effective and less invasive. The present invention satisfies these needs and provides related advantages as well.

### SUMMARY OF THE INVENTION

Accordingly, the primary objective of the present invention is to overcome the limitations of the prior art.

Another object of the invention is to provide methods for treating individuals afflicted with sensitive gums, gingivitis and related conditions of the periodontum of the oral cavity.

It is another object of the present invention to provide a pharmaceutical composition comprising a pharmaceutically acceptable carrier effective in treating sensitive gums, gingivitis and related conditions of the periodontum of the oral cavity.

It is another object of the present invention to provide a method for administering such a composition to an affected region.

It is a further object of the present invention to provide a novel composition and method for treating sensitive gums, gingivitis and related conditions of the periodontum of the oral cavity.

It is another object of the present invention to provide a more effective composition and method for treating sensitive gums, gingivitis and related conditions of the periodontum of the oral cavity.

It is a further object of the present invention to provide a composition and method for treating the mouth sores, swollen gums and tenderness that are associated with gingivitis and periodontal disease, but also preventing the development of the pellicle that develops on the exposed portion of the teeth.

In brief, such stated objects and advantages of the invention are achieved by the application, either in combination or alone, of currently available vitamin E (d-alpha-tocopheryl acetate or dl-alpha-tocopheryl acetate). In one preferred embodiment, a composition comprising between 17,500 I.U. and 56,000 I.U. of vitamin E oil is applied daily to the gumline area of the periodontum affected with gingivitis. However, a composition containing an effective amount of vitamin E oil can be used to treat the same, for example greater than 7,000 I.U. With the composition and method of the instant invention, augmented effectiveness has been observed in the treatment of gingivitis and periodontal disease.

Such stated objects and advantages of the invention are only examples and should not be construed as limiting the present invention. These and other objects, features, aspects, and advantages of the invention herein will become more apparent from the following detailed description of the embodiments of the invention when taken in conjunction with the claims that follow.

### DETAILED DESCRIPTION OF THE PREFERRED EMBODIMENTS

The instant invention relates to a composition and method for treating periodontal disease in general and gingivitis specifically. Although the present invention discloses a preferred method of treating gingivitis by the topical application of the present composition, it is to be understood that the instant invention is not limited thereby and that a combination of applications, i.e. oral or topical, may also be used without departing from the essence of the instant invention as claimed. Topical application as used herein means to apply to the surface of the periodontum of the oral cavity affected by the ailment, e.g. gingivitis.

The percentages and ratios used hereinafter are by weight of the total composition unless specifically designated otherwise. The composition of the present invention comprises vitamin E, however, the composition may also include additional ingredients that do not alter the effectiveness of the combination and do not depart from the essence of the instant invention and, may in fact, augment the effectiveness of the composition of the essential ingredients.

The term “effective amount” is to be understood as meaning an amount of an active ingredient needed to achieve a desired therapeutic or cosmetic effect. For example, in a pharmaceutical composition of the invention an effective amount the composition comprising vitamin E acetate is an amount that is sufficient to achieve an improvement in the condition of the periodontum. In a cosmetic composition, an effective amount is an amount that causes an improvement in the appearance of the periodontum.

Chemically, natural vitamin E is d-alpha-tocopherol. The molecular formula for natural vitamin E is C₂₉H₅₀O₂. It has the following structural formula:

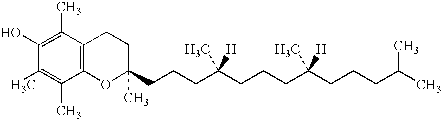

Chemically, synthetic vitamin E is dl-alpha-tocopherol. The molecular formula for synthetic vitamin E is C₂₉H₅₀O₂. It has the following structural formula:

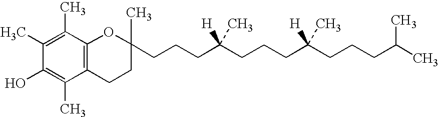

The term “vitamin E” as used in this specification and claims is meant to include not only d-alpha-tocopherol and dl-alpha-tocopherol, but also those analogs and derivatives of vitamin E which are solubilized in the liquid compositions described herein and which have therapeutic activity when topically applied. Vitamin E is employed in the compositions in a therapeutically effective amount. The actual concentration of vitamin E may vary, depending on the nature and degree of the disorders being treated, and whether the drug is being administered for therapeutic or prophylatic purposes. An international unit of vitamin E is generally considered to be equal to 1 milligram of standard dl-alpha-tocopheryl acetate (the acetate ester of synthetic source dl-alpha-tocopherol) or 1 milligram of standard d-alpha-tocopheryl acetate (the acetate ester of natural source d-alpha-tocopherol). The compositions advantageously comprise vitamin E at least greater than 7,000 I.U., and in one preferred embodiment may range between 7,000 to 28,000 I.U. For purposes of illustration, and not limitation, in the examples detailed hereinafter, the vitamin E used was manufactured and distributed by Walgreen Co., located in Deerfield, Ill. 60015-4681 and sold under the trademark WALGREENS NATURE'S FINEST VITAMIN E SKIN OIL; and Colonial Dámes Co. Ltd., located in Los Angeles, Calif. 90022-0022 and sold under the trademark COLONIAL DÁMES VITAMIN E.

### EXAMPLE I

The aim of this example is for purposes of illustration and not limitation. This example demonstrates the effectiveness of the topical application of a composition comprising vitamin E at a concentration of 28,000 I.U. Two to three drops of vitamin E oil was topically applied to the oral cavity of one test subject afflicted with swollen and painful gums for a period of six months. The test subject applied the vitamin E oil to his gums once per evening and did not complain of irritation, discomfort or side effects.

It was noted that the test subject's gingivitis improved through the use of the vitamin E gum wash of the instant composition. The swollen gums had also improved in a shorter duration by the application of the vitamin E gum wash. Vitamin E forms a thin coating or film on the oral substrate and this coating or film acts as a prophylaxis which reduces or prevents new plaque formation for a period after use.

While there are is a specific concentration of vitamin E set forth above, it is to be understood that varying concentrations of the agents can also be used. Therefore, the invention is not limited by the specific concentration listed above. Furthermore, it is to be understood that analogues of the present compounds that are known in the art to have similar functions, may also be used in combination with the active ingredients listed above to augment the effectiveness thereof.

While the above description is very specific, it should not be construed as a limitation on the scope of the invention, but rather as an exemplification of preferred embodiments thereof. Many other variations are possible without departing from the essential spirit of this invention. Accordingly, the scope of the invention should be determined not by the embodiment illustrated, but by the claims and their legal equivalents.

1. A composition for the prophylaxis and treatment of inflammatory diseases of the periodontum, the composition comprising:
vitamin E.

2. The composition of claim 1, wherein said vitamin E is dl-alpha-tocopheryl acetate.

3. The composition of claim 1, wherein said vitamin E is d-alpha-tocopheryl acetate.

4. The composition of claim 1, wherein the concentration of said vitamin E ranges from about 7,000 I.U. to 56,000 IU.

5. The composition of claim 1, wherein the concentration of said vitamin E ranges from about 28,000 I.U. to 56,000 I.U.

6. The composition of claim 1, wherein the concentration of said vitamin E is about 28,000 I.U.

7. The composition of claim 1, wherein the concentration of said vitamin E is about 56,000 I.U.

8. A composition for the prophylaxis and treatment of inflammatory diseases of the periodontum comprising:
a therapeutically effective amount of vitamin E.

9. The composition of claim 8, wherein said vitamin E is d-alpha-tocopheryl acetate.

10. The composition of claim 8, wherein the concentration of said vitamin E present in said composition ranges from about 7,000 I.U. to 56,000 I.U.

11. The composition of claim 8, wherein the concentration of said vitamin E present in said composition ranges from about 28,000 I.U. to 56,000 I.U.

12. The composition of claim 8, wherein the concentration of said vitamin E present in said composition is about 28,000 I.U.

13. The composition of claim 8, wherein the concentration of said vitamin E present in said composition is about 56,000 I.U.

14. (canceled)

15. (canceled)

16. (canceled)

17. (canceled)

18. (canceled)

19. (canceled)

20. (canceled)
